(19) United States
(12) Patent Application Publication
(10) Pub. No.: US 2002/0119168 A1
(43) Pub. Date: Aug. 29, 2002

(54) Therapeutic agent delivery

(76) Inventors: Edward M. Rudnic, N. Potomac, MD (US); James D. Isbister, Potomac, MD (US)

Correspondence Address: Elliot M. Olstein, Esq., c/o Carella, Byrne, Bain, Gilfillan, Cecchi, Stewart & Olstein, 6 Becker Farm Road, Roseland, NJ, 07068, US

(21) Appl. No.: 09/788,965

(22) Filed: Feb. 20, 2001

(51) Int. Cl. A61K 9/00

(52) U.S. Cl. 424/400

(57) ABSTRACT

A therapeutic agent delivery regimen by injection in at least two delivery pulses and no more than thirty-two delivery pulses (preferably by spaced injections) in a period of less than eleven hours that provides the daily dosage for the therapeutic agent.

This invention relates to the delivery of a therapeutic agent, and more particularly to the delivery of a therapeutic agent by injection.

The treatment of a patient with a therapeutic agent by injection is generally known in the art. In general, such treatment is effected by providing an injection once a day, or in some cases twice a day.

The present invention is directed to delivery of a therapeutic agent by injection wherein the therapeutic agent is preferably an antibiotic, an anti-fungal, an anti-neoplastic agent or an antiviral agent.

In accordance with the invention, there is provided a regimen for treating a patient with a therapeutic agent wherein the therapeutic agent is administered by injection, with the daily dosage being delivered over a period that is less than eleven hours (which period is measured from the first injection), and wherein there are at least two delivery pulses, and no more than thirty-two delivery pulses during a period of less than eleven hours, and preferably a period of less than eight hours. As used herein, “delivery pulses” means and may be accomplished by at least two spaced injections with periods between such spaced injections wherein essentially no therapeutic agent is injected into the host or alternatively, between the spaced injections, therapeutic agent is continuously injected in an amount different than that which is injected in the spaced injections. In addition, at least two delivery pulses can be achieved by continuous injection of the agent at one dosage, followed by continuous injection at a different dosage. In such a case there is a first continuous delivery pulse over a period of time, followed by a second continuous delivery pulse over a period of time. Thus, for example, in the latter case, there can be an initial injection wherein the therapeutic agent is continuously administered over a period of time followed by an increase in the dosage of the therapeutic agent that is administered by injection over a period of time whereby in effect there are two delivery pulses even though there may be continuous administration of the therapeutic agent.

In one embodiment, in less than an eleven hour period, there is at least two spaced injections of the therapeutic agent and generally no more than thirty-two spaced injections of the therapeutic agent. There may or may not be a continuous injection of the agent between the spaced injections and if there is such a continuous injection, the dosage of the agent is less than or more than the spaced injections. In a preferred embodiment, there is no injection of agent between the spaced injections.

In one preferred embodiment wherein there are spaced injections of the therapeutic agent, up to about sixty percent, and preferably up to about fifty percent of the dosage that is to be injected in a period of less than eleven hours is injected during the first four hours of such period.

In one embodiment, there is provided two injections in less than a six hour period. In another there is provided no more than six injections preferably in less than six hours. In a further embodiment there is provided at least four injections preferably over less than 6 hours.

In a preferred embodiment, the delivery pulses are accomplished by spaced injections of the therapeutic agent in a pharmaceutically acceptable carrier. There are at least two and no more than 32 spaced injections, all of which are delivered within 11 hours and preferably within 8 hours of the first injection. The daily dosage is delivered within such eleven or eight hour period and the spaced injections provide for at least 75%, preferably at least 90% and more preferably at least 100% of the agent that is to be delivered.

The therapeutic agent may be injected by any procedures known in the art. In a preferred embodiment, the therapeutic agent may be injected by use of a controlled pump of a type known in the art for injecting pharmaceutical products.

Alternatively, the regimen of the invention may be employed in a hospital wherein controlled injections are administered by use of a catheter. Injections can be made into any body structure, organ or blood vessel, such as intravenous, intramuscular, subcutaneous, intradermal, intrathecal, intraperitoneal, intraarticular, intraocular, or other routes of injectable delivery.

In accordance with the invention by employing delivery pulses for injecting the therapeutic agent in a period that is less than eleven hours and preferably less than eight hours, there is provided distinct maximum serum concentration pulses of the therapeutic agent in the blood of the patient in a period of less than 11 hours. In a preferred embodiment, such distinct Cmax pulses occur in a period of less than eight hours and preferably within a period of six hours.

In accordance with a preferred embodiment, all of the Cmax pulses are achieved in a period of less than 11 hours, preferably less than eight hours, and such pulses provide the daily dosage of the therapeutic agent; i.e., the therapeutic agent is injected in at least two delivery pulses within eleven hours and there is no further administration over the remainder of a twenty-four hour period.

All or a portion of the delivery pulses of the therapeutic agent delivered by spaced injections may be the same or different dosages of the therapeutic agent.

In general at a minimum each spaced injection provides at least 5% of the total daily dosage of the therapeutic agent.

It is to be understood that each delivery pulse may include one or more different therapeutic agents (for example two or more different antibiotics), and each delivery pulse may contain the same or different therapeutic agents (for example, one delivery pulse may contain two or more antibiotics and one may contain only one of the two or more antibiotics).

As hereinabove indicated the therapeutic agent is preferably an antibiotic or an anti-viral agent or an anti-fungal agent or an anti-neoplastic agent.

The following are representative examples of some antifungals that can be employed in the invention: amphotericin B, flucytosine, fluconazole, griseofulvin, miconazole nitrate, terbinafine hydrochloride, ketoconazole, itraconazole, undecylenic acid and chloroxylenol, ciclopirox, clotrimazole, butenafine hydrochloride, nystatin, naftifine hydrochloride, oxiconazole nitrate, selenium sulfide, econazole nitrate, terconazole, butoconazole nitrate, carbol-fuchsin, clioquinol, methylrosaniline chloride, sodium thiosulfate, sulconazole nitrate, terbinafine hydrochloride, tioconazole, tolnaftate, undecylenic acid and undecylenate salts (calcium undecylenate, copper undecylenate, zinc undecylenate).

The following are representative examples of some antivirals that may be used in the invention: Acyclovir, Amantadine, Amprenavir, Cidofovir, Delavirdine, Didanosine, Famciclovir, Foscarnet, Ganciclovir, Indinavir, Interferon, Lamivudine, Nelfinavir, Nevirapine, Palivizumab, Penciclovir, Ribavirin, Rimantadine, Ritonavir, Saquinavir, Stavudine, Trifluridine, Valacyclovir, Vidarabine, Zalcitabine, Zidovudine.

The following are representative examples of agents for the treatment of cancer that may be used in accordance with the invention: carboplatin, busulfan, cisplatin, thiotepa, melphalan hydrochloride, cyclophosphamide, ifosfamide, chlorambucil, mechlorethamine hydrochloride, carmustine, lomustine, streptozocin, polifeprosan 20, dexrazoxane, dronabinol, granisetron hydrochloride, fluconazole, erythropoietin, octreotide acetate, pilocarpine hydrochloride, etidronate disodium, pamidronate disodium, allopurinol sodium, amifostine, filgrastim, mesna, ondansetron hydrochloride, dolasetron mesylate, leucovorin calcium, sargramostim, levamisole hydrochloride, doxorubicin hydrochloride, idarubicin hydrochloride, mitomycin, daunorubicin citrate, plicamycin, daunorubicin hydrochloride, bleomycin sulfate, mitoxantrone hydrochloride, valrubicin, dactinomycin, fludarabine phosphate, cytarabine, mercaptopurine, thioguanine, methotrexate sodium, cladribine, floxuridine, capecitabine, anastrozole, bicalutamide, tamoxifen citrate, testolactone, nilutamide, methyltestosterone, flutamide, toremifene citrate, goserelin acetate, estramustine phosphate sodium, ethinyl estradiol, esterified estrogen, leuprolide acetate, conjugated estrogens, megestrol acetate, aldesleukin, medroxyprogesterone acetate, dacarbazine, hydroxyurea, etoposide phosphate, megestrol acetate, paclitaxel, etoposide, teniposide, trastuzumab, rituximab, vinorelbine tartrate, denileukin diftitox, gemcitabine hydrochloride, vincristine sulfate, vinblastine sulfate, asparaginase, edrophonium chloride, bacillus calmette and guerin, irinotecan hydrochloride, pegaspargase, docetaxel, interferon alfa-2a, recombinant, tretinoin, porfimer sodium, interferon alfa-2b, recombinant, procarbazine hydrochloride, topotecan hydrochloride, altretamine, fluorouracil, prednisolone sodium phosphate, cortisone acetate, dexamethasone, dexamethasone sodium sulfate, dexamethasone acetate, hydrocortisone sodium phosphate, hydrocortisone, prednisolone, methylprednisolone sodium succinate, betamethasone sodium phosphate, betamethasone acetate, letrozole, mithramycin, mitotane, pentostatin, perfosfamide, raloxifene.

The following are representative examples of some antibiotics: Cefadroxil, cefazolin, cephalexin, cephalothin, cephapirin, cephacelor, cephprozil, cephadrine, cefamandole, cefonicid, ceforanide, cefuroxime, cefixime, cefoperazone, cefotaxime, cefpodoxime, ceftaxidime, ceftibuten, ceftizoxime, ceftriaxone, cefepime, cefmetazole, cefotetan, cefoxitin, loracarbef, imipenem, erythromycin (and erythromycin salts such as estolate, ethylsuccinate, gluceptate, lactobionate, stearate), azithromycin, clarithromycoin, dirithromycin, troleanomycin, penicillin V, penicillin salts, and complexes, methicillin, nafcillin, oxaciliin, cloxacillin, dicloxacillin, amoxicillin, amoxicillin and clavulanate potassium, ampicillin, bacampicillin, carbenicillin indanyl sodium (and other salts of carbenicillin) meziocillin, piperacillin, piperacillin and taxobactam, ticarcillin, ticarcillin and clavulanate potassium, clindamycin, vancomycin, novobiocin, aminosalicylic acid, capreomycin, cycloserine, ethambutol HCI and other salts, ethionamide, and isoniazid, ciprofloxacin, levofloxacin, lomefloxacin, nalidixic acid, norfloxacin, ofloxacin, sparfloxacin, sulfacytine, suflamerazine, sulfamethazine, sulfamethixole, sulfasalazine, sulfisoxazole, sulfapyrizine, sulfadiazine, sulfmethoxazole, sulfapyridine, metronidazole, methenamine, fosfomycin, nitrofurantoin, trimethoprim, clofazimine, co-triamoxazole, pentamidine, gentanicin, netilmicin, amikacin, and trimetrexate.

Numerous modifications and variations of the present invention are possible in light of the above teachings; therefore, within the scope of the appended claims the invention may be practical otherwise therein as particularly described.

What is claimed is:

1. A process for treating a patient with a therapeutic agent, comprising:
treating a patient by injecting into the patient at least one therapeutic agent in at least two and not more than thirty-two delivery pulses in a period of no more than 11 hours, said therapeutic agent being selected from the group consisting of antibiotics, anti-viral agents, anti-fungal agents and antineoplastic agents.

2. The process of claim 1 wherein said delivery pulses are provided by spaced injections.

3. The process of claim 2 wherein between at least a portion of the spaced injections there is essentially no administration of the therapeutic agent.

4. The process of claim 2 wherein between at least a portion of the spaced injections there is continuous injection of the therapeutic agent in a dosage that is different from the dosage of the spaced injections.

5. The process of claim 2 wherein at least a portion of the spaced injections deliver the therapeutic agent in different dosages.

6. The process of claim 1 wherein there is at least four delivery pulses.

7. The process of claim 6 wherein there is no more than six delivery pulses.

8. The process of claim 7 wherein the total dosage of the therapeutic agent is injected in no more than 6 hours.
